(19) United States
(12) Patent Application Publication
(10) Pub. No.: US 2010/0172982 A1
(43) Pub. Date: Jul. 8, 2010

(54) SUSTAINED RELEASE FORMULATIONS OF DIVALPROEX SODIUM

(75) Inventors: Veera Babu Taduri, Baroda (IN); Deepak Gondaliya, Baroda (IN); Vimal Kaneria, Baroda (IN)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: SUN PHARMACEUTICAL INDUSTRIES LIMITED, Mumbai (IN)

(21) Appl. No.: 12/601,454

(22) PCT Filed: May 23, 2008
(86) PCT No.: PCT/IN2008/000328

§ 371 Date: Jan. 21, 2010

(30) Foreign Application Priority Data
May 23, 2007 (IN) 964/MUM/2007

(51) Int. Cl. A61K 9/24; A61K 31/19; A61P 25/06; A61P 25/08; A61P 25/18

(52) U.S. Cl. 424/468; 514/578

(57) ABSTRACT

A sustained release tablet formulation comprising an inner phase comprising a mixture of divalproex or its pharmaceutically acceptable salt and a hydrophobic agent, and an outer phase comprising a hydrophilic polymer, wherein the hydrophobic agent is present in amount ranging from 6.3% to about 8.3% by weight of the formulation.

### FIELD OF THE INVENTION

The present invention relates to sustained release tablet formulations comprising divalproex sodium for oral administration, a process for preparing such formulations and a method of administering to a patient in need thereof.

### BACKGROUND OF THE INVENTION

Valproic acid, or 2-propylpenatnoic acid and its salts and derivatives are effectively used in the treatment of mania, migraine and epilepsy. After ingestion, the free acid dissociates to the valproate ion within the gastrointestinal tract. The valproate ion is absorbed and produces the desired therapeutic effect.

Divalproex sodium is a stable co-ordination compound comprised of sodium valproate and valproic acid in a 1:1 molar relationship and formed during the partial neutralization of valproic acid with 0.5 equivalent of sodium hydroxide. It is described as a stable crystalline solid and is designated as sodium hydrogen bis(2-propylpentanoate). Divalproex is indicated for the treatment of patients with complex partial seizures, as well as for the treatment of mania associated with bipolar disorders and the prophylaxis of migraine headaches.

Divalproex sodium is commercially available in United States of America as delayed release capsules under the brand name Depakote®, and as extended release or sustained release tablets under the brand name Depakote® ER. The approved Depakote® ER tablets contain Divalproex sodium in a once-a-day extended release formulation equivalent to 250 mg and 500 mg of valproic acid. The inactive ingredients in the compositions include, hypromellose, lactose, microcrystalline cellulose, polyethylene glycol, potassium sorbate, propylene glycol, silicon dioxide, triacetin, FD&C Blue No. 1, iron oxide and polydextrose.

It has been recognized by those skilled in the art that both valproic acid and sodium valproate are difficult to formulate into solid oral dosage forms. Valproic acid and its derivatives are either liquids or liquefy and become sticky. Further, most of them are extremely hygroscopic in nature and therefore pose problems while manufacture of pharmaceutical compositions.

Valproic acid and its derivatives also suffer from another drawback of relatively short elimination half-life. For example in case of valproic acid, a short half-life of between six and seventeen hours and between four and fourteen hours has been reported in adults and children, respectively. This necessitates frequent dosing to maintain a reasonably stable plasma concentration of the drug. The resulting inconvenience to the patient often results in reduced patient compliance with the dosing regimen. Moreover, widely fluctuating plasma concentrations of the drug may result in availability of less or more than the therapeutic amounts of the drug. The reduced dose of drug may result in ineffectiveness of the therapy, while the elevated dose of drug may result in side effects, for example, dizziness, alopecia, etc., as described in Physicians Desk Reference (“PDR”), 52^{nd} Edition, pages 421-437 (2000).

To overcome the above mentioned problems, a significant amount of research work has been directed towards developing sustained release formulations of divalproex sodium, and other valproate compounds to decrease the dosing frequency and to provide a stable plasma concentration of the drug for extended periods of time.

U.S. Pat. No. 6,419,953 (the '953 patent) discloses a controlled release tablet dosage form comprising a valproate compound such as divalproex sodium. The controlled release tablets dosage form comprises a hydrophilic matrix containing a mixture of valproate compound, hydroxypropyl methylcellulose, lactose, microcrystalline cellulose and silicon dioxide having an average particle size ranging between about 1 micron and about 10 microns. The disclosure in the patent particularly requires a hydrophilic polymer to control the release of divalproex sodium.

U.S. application Ser. No. 10/900,415, published as US20060024361, discloses controlled release formulations comprising divalproex sodium that employ a combination of super-disintegrants and a water soluble and/or water insoluble polymers to control the release. The application require the dosage form to be present as a monophasic unit, and the formulations are prepared by a wet granulation process.

U.S. Pat. No. 5,169,642 (the '642 patent) discloses sustained release dosage form comprising divalproex sodium. The sustained release dosage form preparation, comprises divalproex sodium formulated into granules and then coated with a coating composition comprising ethylcellulose and/or methacrylic methylester in an organic solvent. The coated drug granules are further admixed with a viscosity agent and compressed into tablets. As the process involve coating with organic solvents which are either environmentally unsound due to the release of solvent in to the atmosphere or expensive due to the cost of maintaining solvent recovery systems for such process, hence this process is generally not preferred.

It has now been found, a sustained release tablet formulations of divalproex sodium, which formulations on ingestion orally by healthy human subjects produce a desired bioavailability as measured by Cₘₐₓ, AUC₀₋ₜ, and AUC_{0-∞} values when compared with the marketed Deapakote® ER (Divalproex sodium extended release tablets) formulations of Abbott.

### SUMMARY OF THE INVENTION

In one aspect of the invention there is provided a sustained release tablet formulation comprising an inner phase comprising a mixture of divalproex or its pharmaceutically acceptable salt and a hydrophobic agent, and an outer phase comprising a hydrophilic polymer, wherein the hydrophobic agent is present in amount ranging from about 6.3% to about 8.3% by weight of the formulation.

In another aspect of the invention there is provided a sustained release tablet formulation comprising about 42.0% to about 44.0% w/w of Divalproex sodium, about 6.3% to about 8.3% w/w of hydrophobic agent, about 13.5% to about 15.5% w/w of a hydrophilic polymer and a pharmaceutically acceptable excipient, wherein the hydrophobic agent is hydrogenated castor oil and the hydrophilic polymer is hydroxypropylmethylcellulose whose 2% w/v aqueous solution has a viscosity in the range 80,000 to 120,000 mPas at 20° C.

### DETAILED DESCRIPTION OF THE INVENTION

The present invention provides a sustained release tablet formulation comprising an inner phase comprising a mixture of divalproex or its pharmaceutically acceptable salt and a hydrophobic agent, and an outer phase comprising a hydrophilic polymer wherein the hydrophobic agent is present in amount ranging from about 6.3% to about 8.3% by weight of the formulation.

The sustained release tablet formulation of the present invention comprising Divalproex, a hydrophobic agent and a hydrophilic polymer, wherein the ratio of hydrophobic agent to hydrophilic polymer is from about 1:1.7 to about 1:2.5.

The sustained release tablet formulation of the present invention comprises divalproex sodium, a hydrophobic agent and a hydrophilic polymer, wherein the divalproex sodium and hydrophobic agent are melt granulated, mixed with the hydrophilic polymer and wet granulated.

In an embodiment, the sustained release tablet formulations of Divalproex sodium of the present invention provides a sustained release of valproate ion when the dosage form is orally administered to human patients, preferably providing a therapeutic effect for about 24 hours after administration.

In another embodiment, the sustained release tablet formulations of the present invention provides a method of treating human patients in need of Divalproex sodium sustained release therapy, comprising orally administering to a human patient an effective amount of a sustained release Divalproex sodium oral solid dosage form prepared in accordance with the present invention on a once a day basis.

In another embodiment, the sustained release tablet formulations of the present invention provides a method of treating complex seizures, mania associated with bipolar disorders, and/or migraine headaches in humans comprising orally administering an effective amount of a sustained release Divalproex sodium oral solid dosage form prepared in accordance with the present invention to a human patient on a once a day basis.

The sustained release tablet formulation of the present invention comprising an inner phase comprising a mixture of divalproex or its pharmaceutically acceptable salt and a hydrophobic agent; and an outer phase comprising a hydrophilic polymer, wherein the hydrophobic agent is present in amount ranging from about 6.3% to about 8.3% by weight of the formulation and the tablet composition is manufactured at controlled environmental conditions of temperature of less than about 25° C. and relative humidity of less than about 30%.

“Cₘₐₓ” as used herein, means maximum plasma concentration of the valproate ion, produced by the ingestion of composition comprising Divalproex sodium.

“Tₘₐₓ” as used herein, means time to the maximum observed plasma concentration (Cₘₐₓ) described above.

“AUC” as used herein, means area under the plasma concentration vs time curve, as calculated by the trapezoidal rule over the complete 24 hour interval for all the formulations.

“AUC₀₋ₜ” as used herein, means area under the plasma concentration vs time curve from 0 hours to the time (t) of last sample collected.

“AUC_{0-∞}” as used herein, means area under the plasma concentration vs time curve from 0 hours to infinity.

The term “desired bioavailability as measured by Cₘₐₓ, AUC₀₋ₜ and AUC_{0-∞} values” as used herein, indicates that the 90% confidence interval of the relative mean of Cₘₐₓ, AUC₀₋ₜ and AUC_{0-∞} values of the formulation of the present invention to the relative mean of Cₘₐₓ, AUC₀₋ₜ and AUC_{0-∞} values of a sustained release formulation comprising an equivalent dose of Divalproex sodium sold under the brand name Depakoate® ER tablets, are within 80-125%.

According to one embodiment, there is provided a process for providing a sustained release tablet formulation of Divalproex sodium having a desired bioavailability as measured by Cₘₐₓ, AUC₀₋ₜ and AUC_{0-∞} values, said process comprising:

1. Selecting a suitable hydrophobic agent and a hydrophilic polymer and preparing a sustained release tablet formulation comprising:
a. about 42% to about 44% w/w of Divalproex sodium;
b. about 6.3% to about 8.3% w/w of hydrophobic agent; and
c. about 13.5% to about 15.5% w/w of a hydrophilic polymer;
d. optionally, other pharmaceutically acceptable excipients;
   2. Administering the tablet formulation to healthy human subjects and measuring the bioavailability.
   3. Decreasing or increasing the content of the hydrophobic agent and measuring the bioavailability.
   4. Repeat step 3 until the desired bioavailability is obtained;
   wherein to decrease the bioavailability, the content of the hydrophobic agent is increased and to increase the bioavailability, the content of hydrophobic agent is decreased.

The Divalproex sodium that is used in the present invention, is a stable co-ordination compound comprised of sodium valproate and valproic acid in a 1:1 molar relationship and formed during the partial neutralization of valproic acid with 0.5 equivalent of sodium hydroxide. The amount of Divalproex sodium that may be present in the formulation is from about 42% to about 44% w/w of the formulation.

The hydrophobic agents that may be used in the present invention include water insoluble polymers and waxes. Examples of suitable water insoluble hydrophobic polymers include acrylates, cellulose derivatives such ethylcellulose or cellulose acetate, polyethylene, methacrylates, acrylic acid copolymers, high molecular weight polyvinylalcohols, stearyl alcohol, glyceryl palmitostearte, glyceryl monostearate, waxes such as carnauba wax, beeswax candelilla wax, microcrystalline wax, ozokerite wax, paraffin waxes, castorwax (hydrogenated castor oil) and hydrogenated vegetable oil (Lubritab) and mixtures thereof. Preferably, the hydrophobic agent is used in amount ranging from about 6.3% to about 8.3% w/w of the formulation.

In a preferred embodiment of the present invention the hydrophobic agent is a wax, preferably hydrogenated castor oil. The hydrogenated castor oil sold under the brand name Cutina HR PH from Cognis, is the most preferred. Preferably it is used in amount ranging from about 6.3% to about 8.3% w/w of the formulation.

The hydrophilic polymers that may be used in the present invention include water soluble hydrophilic polymers. Examples of suitable water soluble polymers include polyvinylpyrrolidine, hydroxypropyl cellulose, hydroxypropylmethyl cellulose, methyl cellulose, vinyl acetate copolymers, polysaccharides (such as alignate, xanthum gum, etc.) polyethylene oxide, methacrylic acid copolymers, maleic anhydride/methyl vinyl ether copolymers and derivatives, and mixtures thereof.

In a preferred embodiment of the present invention, the hydrophilic polymer is hydroxypropyl methylcellulose. Various grades of hydroxypropyl methylcellulose (available from Dow Chemical, U.S.A under the METHOCEL trademark) may be used in the present invention. The grades commercially available are categorized depending upon the chemical substitution and hydration rates, and may be used in the compositions of the present invention. Hydroxypropyl methylcellulose having a methoxy content of 19-24% and hydroxypropyl content of 7-12% with a fastest relative rate of hydration is available commercially under the brand name of Methocel Grade K. Hydroxypropyl methylcellulose with 28-30% methoxy content and 7-12% of hydroxypropyl content with a faster relative hydration rate as compared to the above grade is available commercially under the brand name of Methocel Grade E. Hydroxypropyl methylcellulose with 27-30% methoxy content and 4.0-7.5% of hydroxypropyl content with a slow relative hydration rate is available as Methocel F grade and that with 27.5-31.5% methoxy content and 0% hydroxypropyl content and with slowest rate of hydration is available as Methocel Grade A.

In a more preferred embodiment of the present invention, hydroxypropyl methylcellulose, which is commercially available as METHOCEL K100M is used. A 2% w/v aqueous solution of METHOCEL K100M, has a viscosity in the range 80,000 to 120,000 mPas at 20° C. It may be used in the amounts ranging from about 13.5% to about 15.5% w/w of the formulation.

The sustained release tablet formulation of the present invention also includes pharmaceutically acceptable inert excipients well known in the art for manufacturing of solid dosage forms. These are added to ease the manufacturing process as well as to improve the performance of the dosage form. The common excipients include diluents or fillers or bulking agents, binders, lubricants, glidants, coloring agents, plasticizers, granulating aids, flavorants and the like. All excipients are used in a manner known to the persons skilled in the pharmaceutical art, and in amounts conventional in the art.

Diluents are added in order to increase the mass of an individual dose to a size suitable for tablet compression. Suitable diluents include calcium sulfate, calcium phosphate dibasic, calcium phosphate tribasic, microcrystalline cellulose, calcium carbonate, dextrose, spray dried lactose, anhydrous lactose, lactose mono hydrate, mannitol, sorbitol, dextrins, sucrose, starch pregelatinized, mixtures thereof and the like.

Binders are typically added when the manufacture of dosage forms uses a granulation step. Examples of suitable binders include acacia, methyl cellulose, hydroxypropyl methylcellulose, ethyl cellulose, polyvinyl alcohol, pullulan, pregelatinized starch, gelatin, glucose, polyvinylpyrrolidone, sodium alginate and alginate derivatives, agar, mixtures thereof and the like.

Lubricants are usually incorporated into the formulation of solid dosage forms to reduce friction between the granules and the die wall during compression and ejection. This prevents problems of sticking associated with manufacturing of tablets and also facilitates easy ejection of the tablets form the punches. Examples of suitable lubricants include talc, silicon dioxide (carbosil), amorphous silicon dioxide (Syloid 244 EP), stearic acid, vegetable oil, calcium stearate, magnesium stearate, mixtures thereof and the like. The preferred lubricant for the sustained release tablet formulation of the present invention is amorphous silicon dioxide (syloid 244 EP).

Glidants are typically incorporated into the formulations to improve the flow characteristics of the granules. Examples of suitable glidants include talc, corn starch, silicon derivatives, mixtures thereof and the like.

The sustained relase tablet formulation of the present invention may include solvents for use in wet granulation of the composition. Examples of the solvents that may be used include water, alcohol, halogenated solvents and the like, and mixtures thereof. The preferred solvent to be used in the process of granulation of the pharmaceutical composition of the present invention is a mixture of water and isopropyl alcohol.

According to an embodiment, the sustained release tablet formulation of the present invention may include coloring agents that are generally acceptable to Food and Drug Administration (FDA) for use in oral formulations.

According to an embodiment, the sustained release tablet formulations of the present invention may be optionally coated with a pharmaceutically acceptable film coating, e.g., for aesthetic purposes (e.g., including a colorant), for stability purposes (e.g., coated with a moisture barrier coating), for taste masking purposes, etc. The film coating may include film forming polymers and other inert additives like plasticizers, pigments, and the like, generally used in the pharmaceutical art. Examples of the film forming polymers include ethyl cellulose, hydroxypropylmethylcellulose, hydroxypropyl cellulose, methylcellulose, carboxymethyl cellulose, hydroxymethylcellulose, hydroxyethylcellulose, cellulose acetate and the like. Alternatively, commercially available coating compositions comprising film forming polymers marketed under various trade names, such as Opadry® may also be used for coating. It may be noted that none of these film coatings mentioned above, affect release of the Divalproex sodium from the composition in any way.

According to one embodiment, the sustained release tablet formulation of the present invention comprises about 42.0% to about 44.0% w/w of Divalproex sodium, about 6.3% to about 8.3% w/w of hydrophobic agent, about 13.5% to about 15.5% w/w of a hydrophilic polymer and a pharmaceutically acceptable excipient, wherein the hydrophobic agent is hydrogenated castor oil and the hydrophilic polymer is hydroxypropylmethylcellulose whose 2% w/v aqueous solution has a viscosity in the range 80,000 to 120,000 mPas at 20° C.

In another embodiment, the sustained release tablet formulation of the present invention comprises about 42.0% to about 44% w/w Divalproex sodium, about 6.3% to about 8.3% w/w hydrogenated castor oil, about 10% to about 12.5% w/w lactose anhydrous, about 13.5% to about 15.5% w/w hydroxypropylmethylcellulose, about 10.5% to about 13.0% w/w microcrystalline cellulose, about 1.0% to about 3.5% w/w colloidal silicon dioxide, about 1.0% to about 3.5% w/w magnesium stearate, about 2.5% to about 5.0% w/w talc, and about 2.0% to about 4.5% w/w opadry.

In a further embodiment, the sustained release tablet formulation of the present invention comprises about 42.0% to about 44% w/w Divalproex, sodium, about 6.3% to about 8.3% w/w hydrogenated castor oil, about 26.5% to about 29.0% w/w lactose anhydrous, about 13.5% to about 15.5% w/w hydroxypropylmethylcellulose, about 0.5% to about 3.0% w/w silicon dioxide (syloid), about 0.5% to about 2.0% w/w magnesium sterate, about 0.5% to 3.0% w/w talc and about 2.0% to about 4.5% w/w opadry.

In an embodiment, the sustained release tablet formulation of the present invention, wherein the said tablet when measured in a type 2 dissolution apparatus, paddle, at 100 rpm, at a temperature of 37±0.5° C., in 500 ml of 0.1N HCl for the first 45 minutes, followed by 900 ml of 0.05M phosphate buffer containing 75 mM sodium lauryl sulphate at pH 5.5, for the remainder of the testing period, exhibits an in vitro dissolution profile as follows:
i) No more than about 30% total valproate is released after 3 hours of measurement in the said apparatus;
ii) From about 40% to about 75% of total valproate is released after 9 hours of measurement in said apparatus;
iii) From about 80% to about 90% of total valproate is released after 12 hours of measurement in said apparatus;
iv) Not less than 95% of total valproate is released after 16 hours of measurement in said apparatus.

In another embodiment, the sustained release tablet formulation of divalproex sodium of the present invention, when administered orally to healthy human subjects produce a desired bioavailability as measured by Cₘₐₓ, AUC₀₋ₜ and AUC_{0-∞} values when compared with the marketed Deapakote® ER (Divalproex sodium extended release tablets) formulations of Abbott.

The sustained release tablet formulations of the present invention are prepared by melt granulation followed by wet granulation process. The Divalproex sodium particles are initially admixed with a hydrophobic agent by a process of melt granulation. The granules of divalproex sodium obtained are then mixed with a hydrophilic polymer and wet granulated to obtain a sustained release granules which are then compressed to form a solid dosage form.

In a preferred embodiment, the process of preparation comprises the steps of blending Divalproex sodium, hydrophobic agent and optionally pharmaceutically inert excipient; melt granulating the blend followed by solidifying into a compact mass; breaking the compact mass into granules; blending with a hydrophilic polymer and optionally with other pharmaceutically inert excipients followed by wet granulation using a mixture of water and ispopropyl alcohol to obtain granules; lubricating the granules and compressing the lubricated granules into tablets, and; optionally film coating the tablets with film forming polymer and coating additives.

It will be understood by those of skill in the art that numerous and various modifications can be made without departing from the spirit of the present invention. Therefore, it should be clearly understood that the following examples are illustrative only and are not to be construed to limit the scope of the present invention.

### Comparative Example 1

The Divalproex sodium extended release tablet formulations are prepared as shown in Table 1 below.

TABLE 1

Quantity

| | | |
|---|---|---|
| Ingredients | mg/tablet | % w/w |

Inner Phase

| | | |
|---|---|---|
| Divalproex Sodium | 538.00 | 43.40 |
| Hydrogenated castor oil | 120.00 | 9.68 |
| (Cutina HR PH) | | |

Outer Phase

| | | |
|---|---|---|
| Lactose anhydrous | 125.00 | 10.08 |
| Hydroxypropyl methylcellulose | 180.00 | 14.52 |
| (HPMC K100 M) | | |
| Microcrystalline cellulose | 132.00 | 10.64 |
| PH 101 | | |
| Colloidal silicon dioxide | 30.00 | 2.42 |
| Magnesium stearate | 30.00 | 2.42 |
| Talc | 45.00 | 3.63 |

Film Coat

| | | |
|---|---|---|
| Opadry 03F57509 | 39.60 | 3.19 |
| Total | 1239.60 | 100.00 |

### Process of Manufacture:

Divalproex sodium was milled through a 2 mm screen, knives forward, medium speed using a comminuting mill before loading into the jacketed rapid mixer granulator (RMG) for granulation. Hydrogenated castor oil (Cutina HR PH) was milled through 20 mesh sieve prior to loading into RMG. The blend in the RMG was mixed for 5 minutes at slow/off speed. The blend in the RMG was then heated to approximately to 80°-85° C., at steam pressure range of 0.2-0.6 kg/cm² under continuous mixing and circulating steam in the jacket till granules (lumps formation) were formed. The resulting granules were removed from the RMG and chilled water was circulated through the jacket to cool the RMG. Granules formed were then transferred back to the cooled RMG and mixed. Drug granules obtained from RMG were sifted through 20 mesh sieve and over sized granules were milled through 10 mm screen, knives forward, slow speed using a comminuting mill. The blend of anhydrous lactose, hydoxypropyl methylcellulose, and microcrystalline cellulose PH 101, were sifted through 40 mesh sieve and transferred to RMG containing drug granules. The wet granulation was done by adding isopropyl alcohol to the blend in the RMG till the granules were formed. The granules were then milled in a comminuting mill having 8 mm sieve at slow speed and knives forward. The granules were air dried using Fluid bed drier at a temperature of about 55° C. to 65° C. for 15 minutes, till the moisture content was not more than 0.5%. The dried granules were sifted through 16 mesh sieve. Talc and magnesium stearate, which were sifted through 60 mesh sieve, were added to the granules obtained. Silicon dioxide sifted through 40 mesh sieve, was added to the blend obtained and blended with above granules for 15 minutes. The tablets were compressed using 20×10 mm oval punches at a compression force between 10.0 to 16.0 Kilo pound (Kp). The tablets were then film coated with a dispersion of opadry to a weight gain of 3.0%.

### Invitro Dissoloution Study

Invitro dissolution tests were conducted for Divalproex sodium extended release tablet formulations prepared in comparative Example 1 and compared with the marketed Depakote®ER tablets (500 mg) of Abbott laboratories. The testing was performed using USP Type 2 dissolution apparatus, operating at 37±0.5° C. with a paddle rotating speed of 100 rpm. The tablets were tested, in 500 ml of 0.1N hydrochloric acid for first 45 minutes, followed by 900 ml of 0.05 M phosphate buffer containing 75 mM sodium lauryl sulphate at pH 5.5. The results are summarized in Table 2 below:

TABLE 2
Comparative dissolution profile of Divalproex sodium extended
release tablets (equivalent to 500 mg of valproic acid) of
Comparative Example 1 and Depakote ® ER tablets, 500 mg.

Cumulative percentage (%) release of valproic acid

| Time | | Depakote ® ER tablet |
|---|---|---|
| (hr) | Comparative Example 1 | (500 mg) |
| 3 | 25.25 | 25.39 |
| 9 | 61.82 | 68.59 |
| 12 | 72.30 | 96.00 |
| 16 | 85.02 | 104.19 |

From the above study it is clearly evident that the invitro release of valproic acid is less compared to the Depakoate® ER tablets.

### Bioavailability Study

The bioavailability study of Divalproex sodium extended release tablet formulation of comparative Example 1 was carried out on healthy human male volunteers (n=14). Depakote®ER tablet (500 mg) was used as the reference. Thirteen human volunteers completed the study. The study was conducted according to a single dose, open label, randomized, two treatment; two period and two sequence, comparative and two way crossover study under fed and fasted conditions. The results are shown in Table 3 and Table 4 below.

TABLE 3
Pharmacokinetic parameters obtained through the bioavailability
studies of Divalproex sodium extended release tablets of
comparative Example 1 (test product) and Depakote ® ER
tablets (reference product) in fed condition.

| | Pharmacokinetic Parameter | | | |
|---|---|---|---|---|
| | | AUC₀₋ₜ | AUC_{0-∞} | Tₘₐₓ |
| | Cₘₐₓ (μg/ml) | (μg · hr/ml) | (μg · hr/ml) | (hr) |
| | Fed Condition | | | |
| Divalproex sodium ER | 26.39 | 891.42 | 963.63 | 22.00 |
| tablet (500 mg) of | | | | |
| comparative | | | | |
| Example 1 | | | | |
| Depakote ® ER tablet | 30.02 | 899.47 | 969.66 | 17.50 |
| (500 mg) | | | | |
| 90% confidence | 80.96-95.49 | 89.98-109.16 | 92.89-106.32 | — |
| interval | | | | |
| [Test/Reference] | 87.92 | 99.10 | 99.38 | — |
| Ratio (%) | | | | |

TABLE 4
Pharmacokinetic parameters obtained through the bioavailability
studies of Divalproex sodium extended release tablets
of comparative Example 1 (test product) and Depakote ® ER
tablets (reference) in fasted condition.

| | Pharmacokinetic Parameter | | | |
|---|---|---|---|---|
| | | AUC₀₋ₜ | AUC_{0-∞} | Tₘₐₓ |
| | Cₘₐₓ (μg/ml) | (μg · hr/ml) | (μg · hr/ml) | (hr) |
| | Fasting Condition | | | |
| Divalproex sodium ER | 20.09 | 601.20 | 646.91 | 5.75 |
| tablet (500 mg) of | | | | |
| comparative | | | | |
| Example 1 | | | | |
| Depakote ® ER tablet | 24.13 | 730.12 | 787.79 | 5.50 |
| (500 mg) | | | | |
| 90% confidence | 68.36-99.19 | 64.83-103.03 | 65.36-101.94 | — |
| interval | | | | |
| [Test/Reference] | 82.30 | 81.70 | 81.60 | — |
| Ratio (%) | | | | |

From the above study, it was found that the 90% confidence interval for the test/reference ratio for relative mean of Cₘₐₓ, AUC₀₋ₜ and AUC_{0-∞} values of the tablet formulations of the present invention to the relative mean of Cₘₐₓ, AUC₀₋ₜ and AUC_{0-∞} values of a sustained release composition comprising an equivalent dose of Divalproex sodium sold under brand name Depakote®ER tablet, is less under fasting conditions, while it was found to be comparable under fed conditions.

### Examples 1-2

The divalproex sodium sustained release tablet formulations of the present invention were prepared as shown in Table 5 below.

TABLE 5

| | Quantity | | | |
|---|---|---|---|---|
| | Example 1 | | Example 2 | |
| Ingredients | mg/tablet | % w/w | mg/tablet | % w/w |

Inner Phase

| | | | | |
|---|---|---|---|---|
| Divalproex Sodium | 538.00 | 43.40 | 538.00 | 43.40 |
| Hydrogenated castor oil | 90.00 | 7.26 | 90.00 | 7.26 |
| (Cutina HR PH) | | | | |

Outer Phase

| | | | | |
|---|---|---|---|---|
| Lactose anhydrous | 140.00 | 11.29 | 344.00 | 27.75 |
| Hydroxypropyl | 180.00 | 14.52 | 180.00 | 14.52 |
| methylcellulose | | | | |
| (HPMC K100 M) | | | | |
| Microcrystalline cellulose | 147.00 | 11.85 | — | — |
| PH 101 | | | | |
| Colloidal silicon dioxide | 30.00 | 2.42 | — | — |
| Silicon dioxide | — | — | 24.00 | 1.93 |
| (Syloid 244 FP) | | | | |
| Magnesium stearate | 30.00 | 2.42 | 12.00 | 0.97 |
| Talc | 45.00 | 3.63 | 12.00 | 0.97 |

Film Coat

| | | | | |
|---|---|---|---|---|
| Opadry 03F57509 | 39.6 | 3.19 | 39.6 | 3.19 |
| Total | 1239.60 | 100 | 1239.60 | 100 |

### Process of Manufacture:

Divalproex sodium was milled through a 2 mm screen, knives forward, medium speed using a comminuting mill before loading into the jacketed rapid mixer granulator (RMG) for granulation. Hydrogenated castor oil (Cutina HR PH) was milled through 20 mesh sieve prior to loading into RMG. The blend in the RMG was mixed for 5 minutes at slow/off speed. The blend in the RMG was then heated to approximately to temperature of 60°-90° C., at steam pressure range of 0.2-0.6 kg/cm² under continuous mixing and circulating steam in the jacket till granules (lumps formation) were formed. The resulting granules were removed from the RMG and chilled water was circulated through the jacket to cool the RMG. Granules formed were then transferred back to the cooled RMG and mixed. Drug granules obtained from RMG were sifted through 20 mesh sieve and over sized granules were milled through 10 mm screen, knives forward, slow speed using a comminuting mill. The blend of anhydrous lactose, hydoxypropyl methylcellulose, and microcrystalline cellulose PH 101, were sifted through 40 mesh sieve and transferred to RMG containing drug granules. The wet granulation was done by adding isopropyl alcohol alone or with a mixture of water and isopropyl alcohol, to the blend in the RMG till the granules were formed. The granules were then milled a in comminuting mill having 8 mm sieve at slow speed and knives forward. The granules were air dried using Fluid bed drier at a temperature of about 55° C. to 65° C. for 15 minutes, till the moisture content is not more than 0.5%. The dried granules were sifted through 16 mesh sieve. Talc and magnesium stearate which were sifted through 60 mesh sieve were added to the granules obtained. Colloidal silicon dioxide or Silicon dioxide (Syloid 244 FP) sifted through 40 mesh sieve, was added to the blend obtained and blended with above granules for 15 minutes. The tablets were compressed using 20×10 mm oval punches at a compression force between 10.0 to 16.0 Kilo pound (Kp). The tablets were then film coated with a dispersion of opadry to gain a weight of 3.0%.

### Example 3

Invitro dissolution tests were conducted for Divalproex sodium extended release tablet formulations of the present invention, shown in Example 1 and compared with the marketed Depakote®ER tablets (500 mg) of Abbott laboratories. The testing was performed using USP Type 2 dissolution apparatus, operating at 37±0.5° C. with a paddle rotating speed of 100 rpm. The tablets were tested in 500 ml of 0.1N hydrochloric acid for first 45 minutes, followed by 900 ml of 0.05 M phosphate buffer containing 75 mM sodium lauryl sulphate at pH 5.5. The results are summarized in Table 6 below.

TABLE 6
Comparative dissolution profile of Divalproex sodium extended
release tablets (equivalent to 500 mg of valproic acid)
of example 1 and Depakote ® ER tablets, 500 mg.

Cumulative percentage (%) release of valproic acid

| Time | | Depakote ® ER tablet |
|---|---|---|
| (hr) | Example 1 | (500 mg) |
| 3 | 28.89 | 25.39 |
| 9 | 72.03 | 68.59 |
| 12 | 85.56 | 96.00 |
| 16 | 98.98 | 104.19 |

From the above study it is clearly evident that the invitro release of valproic acid from the composition of Example 1 are comparable to that of the Depakote®ER tablet (500 mg).

### Example 4

The bioavailability study of Divalproex sodium extended release tablet formulation of Example 1 was carried out on healthy human male volunteers (n=16) using Depakote®ER tablet (500 mg) as the reference. Fifteen human volunteers completed the two way crossover study in fasted condition, while thirteen human volunteers completed the fed condition study. The study was conducted according to a single dose, open label, randomized, two treatment, two period and two sequence, comparative and two way crossover study under fed and fasted conditions. The results are shown in Table 7 and Table 8 below.

TABLE 7
Pharmacokinetic parameters obtained through the bioavailability
studies of Divalproex sodium extended release tablets
of Example 1(test product) and Depakote ® ER
tablets (reference product) in fed condition.

| | Pharmacokinetic Parameter | | | |
|---|---|---|---|---|
| | | AUC₀₋ₜ | AUC_{0-∞} | Tₘₐₓ |
| | Cₘₐₓ (μg/ml) | (μg · hr/ml) | (μg · hr/ml) | (hr) |
| | Fed Condition | | | |
| Divalproex sodium ER | 24.07 | 817.79 | 840.42 | 22.00 |
| tablet of Example 1 | | | | |
| Depakote ® ER tablet | 24.67 | 801.53 | 846.45 | 20.00 |
| (500 mg) | | | | |
| 90% confidence | 80.69-112.61 | 90.79-118.15 | 88.58-119.21 | — |
| interval | | | | |
| [Test/Reference] | 98.8 | 103.60 | 102.80 | — |
| Ratio (%) | | | | |

TABLE 8
Pharmacokinetic parameters obtained through the bioavailability
studies of Divalproex sodium extended release tablets
of Example 1 (test product) and Depakote ® ER
tablets (reference product) in fasting condition.

| | Pharmacokinetic Parameter | | | |
|---|---|---|---|---|
| | | AUC₀₋ₜ | AUC_{0-∞} | Tₘₐₓ |
| | Cₘₐₓ (μg/ml) | (μg · hr/ml) | (μg · hr/ml) | (hr) |
| | Fasting Condition | | | |
| Divalproex sodium ER | 22.51 | 765.14 | 845.36 | 18.00 |
| tablet of Example 1 | | | | |
| Depakote ® ER tablet | 21.30 | 769.19 | 873.26 | 18.00 |
| (500 mg) | | | | |
| 90% confidence | 97.60-114.75 | 90.39-109.38 | 89.21-105.08 | — |
| interval | | | | |
| [Test/Reference] | 105.80 | 99.40 | 96.80 | — |
| Ratio (%) | | | | |

From the above study, it was found that the 90% confidence interval for the test/reference ratio for relative mean of Cₘₐₓ, AUC₀₋ₜ and AUC_{0-∞} values of the composition of the present invention to the relative mean of Cₘₐₓ, AUC₀₋ₜ and AUC_{0-∞} values of a sustained release composition comprising an equivalent dose of Divalproex sodium and hydrophilic polymer was found to be comparable under fed and fasted conditions. Hence, the formulations of Example 1 and Example 2 are bioequivalent to the reference product (Divalproex® ER tablet, 500 mg, Abbott Lab, USA).

The invention having been described, it will be readily apparent to those skilled in the art that further changes and modifications in actual implementation of the concepts and embodiments described herein can easily be made or may be learned by practice of the invention, without departing from the spirit and scope of the invention as defined by the following claims.

1. A sustained release tablet formulation comprising an inner phase comprising a mixture of divalproex or its pharmaceutically acceptable salt and a hydrophobic agent, and an outer phase comprising a hydrophilic polymer, wherein the hydrophobic agent is present in amount ranging from 6.3% to about 8.3% by weight of the formulation.

2. A sustained release tablet formulation as in claim 1, wherein the said divalproex or its pharmaceutically acceptable salt is present in amount ranging from about 42.0% to about 44.0% by weight of the formulation.

3. A sustained release tablet formulation as in claim 1, wherein the said hydrophobic agent is hydrogenated castor oil.

4. A sustained release tablet formulation as in claim 1, wherein the said hydrophilic polymer is a hydroxypropylmethylcellulose, whose 2% w/v aqueous solution has a viscosity in the range 80,000 to 120,000 mPas at 20° C. and is present in an amount ranging from about 13.5% to about 15.5% by weight of the formulation.

5. A sustained release tablet formulation as in claim 1, wherein the ratio of hydrophobic agent to hydrophilic polymer in the formulation is from about 1:1.7 to about 1:2.5.

6. A sustained release tablet formulation comprising about 42.0% to about 44.0% w/w of Divalproex sodium, about 6.3% to about 8.3% w/w of hydrophobic agent, about 13.5% to about 15.5% w/w of a hydrophilic polymer, and a pharmaceutically acceptable excipient, wherein the hydrophobic agent is hydrogenated castor oil and the hydrophilic polymer is a hydroxypropylmethylcellulose, whose 2% w/v aqueous solution has a viscosity in the range 80,000 to 120,000 mPas at 20° C.

7. A sustained release tablet formulation as claim 1, wherein the said tablet when measured in a type 2 dissolution apparatus, paddle rotating at 100 rpm, at a temperature of 37±0.5° C., in 500 ml of 0.1N HCl for the first 45 minutes, followed by 900 ml of 0.05M phosphate buffer containing 75 mM sodium lauryl sulphate at pH 5.5, for the remainder of the testing period, exhibits an in vitro dissolution profile as follows:
a. Not more than about 30% total valproate is released after 3 hours of measurement in the said apparatus;
b. From about 40% to about 75% of total valproate is released after 9 hours of measurement in said apparatus;
c. From about 80% to about 90% of total valproate is released after 12 hours of measurement in said apparatus;
d. Not less than 95% of total valproate is released after 16 hours of measurement in said apparatus.

8. A sustained release tablet formulation as claim 6, wherein the said tablet when measured in a type 2 dissolution apparatus, paddle rotating at 100 rpm, at a temperature of 37±0.5° C., in 500 ml of 0.1N HCl for the first 45 minutes, followed by 900 ml of 0.05M phosphate buffer containing 75 mM sodium lauryl sulphate at pH 5.5, for the remainder of the testing period, exhibits an in vitro dissolution profile as follows:
a. Not more than about 30% total valproate is released after 3 hours of measurement in the said apparatus;
b. From about 40% to about 75% of total valproate is released after 9 hours of measurement in said apparatus;
c. From about 80% to about 90% of total valproate is released after 12 hours of measurement in said apparatus;
d. Not less than 95% of total valproate is released after 16 hours of measurement in said apparatus.
